(19) United States
(12) Patent Application Publication
(10) Pub. No.: US 2025/0216376 A1
(43) Pub. Date: Jul. 3, 2025

(54) METHOD OF ANALYZING MICROPLASTIC PARTICLES IN WATER SYSTEM

(71) Applicant: SK INNOVATION CO., LTD., Seoul (KR)

(72) Inventors: Jae Yang SONG, Daejeon (KR); Yeon Hwa LA, Daejeon (KR); A Rum SIM, Daejeon (KR); Tae Wan KIM, Daejeon (KR); Hye Lin ROH, Daejeon (KR)

(21) Appl. No.: 19/001,568

(22) Filed: Dec. 26, 2024

(30) Foreign Application Priority Data
Dec. 28, 2023 (KR) 10-2023-0195465

(51) Int. Cl. G01N 33/44; G01J 3/453; G01N 1/42; G01N 1/44; G01N 21/65; G01N 30/72

(52) U.S. Cl. CPC G01N 33/442; G01J 3/453; G01N 1/42; G01N 1/44; G01N 21/65; G01N 30/7206; G01N 2223/623

(57) ABSTRACT

Proposed is a method of analyzing microplastic particles in a water system. The method includes providing raw water containing microplastic particles, freezing and thawing the raw water at least once to generate microplastic aggregates in the raw water, recovering the microplastic aggregates, and analyzing the recovered microplastic aggregates. According to the method, the microplastic particles are precipitated in the water system without the use of an additional coagulant, whereby the effect of coagulants on the subsequently recovered microplastic aggregates may be ruled out. In addition, the process configuration is simple because the aggregates can be separated from the supernatant without using any additional treatment process.

### CROSS REFERENCE TO RELATED APPLICATION

The present application claims priority to Korean Patent Application No. 10-2023-0195465, filed Dec. 28, 2023, the entire contents of which are incorporated herein for all purposes by this reference.

### BACKGROUND OF THE DISCLOSURE

### 1. Field of the Disclosure

Embodiments of the present disclosure relate to a method of analyzing microplastic particles in a water system.

### 2. Description of the Related Art

Globally, the use of disposable products, especially plastic disposable products such as polyethylene terephthalate (PET), has been significantly increasing. Accordingly, environmental pollution problems such as microplastic-caused water pollution are emerging. Research and development on methods of analyzing microplastic particles in a water system is also being actively conducted.

In particular, among microplastic particles in a water system, microplastic particles with sizes of several micrometers or more may be separated using a filter and analyzed using conventional analysis techniques. However, nano-sized microplastic particles that cannot be filtered by filters are not easy to separate from the water system. Therefore, research is being actively conducted on techniques to separate and analyze nano-sized microplastic particles from the water system.

### SUMMARY OF THE DISCLOSURE

According to embodiments of the present disclosure, there is provided a simplified and highly accurate method of analyzing microplastic particles contained in a water system. The method is particularly suitable for measuring nano-sized microplastic particles in a water system.

In an embodiment of the present disclosure, a method of analyzing microplastic particles in a water system may include providing raw water (for example, to-be-treated water) containing microplastic particles; freezing and thawing the raw water at least once to generate microplastic aggregates in the raw water; recovering the microplastic aggregates; and analyzing the recovered microplastic aggregates. The term raw water as used here means untreated or partially treated water that contains microplastic particles. For example, the raw water may be water with microplastic particles, or water with microplastic particles and one or more impurities. The impurities may be adhering on the surface of the microplastic particles. The method of analysis of microplastics may be performed at any stage of a water treatment or a water analysis system. In an embodiment, the method may further include removing an impurity on the surface of the microplastic particles in the raw water before the freezing and thawing.

In an embodiment, the removal of the impurity on the surface of the microplastic particles may be performed by one of a strong acid or strong alkali treatment, an ultrasonic treatment, a plasma treatment, an electrochemical oxidation treatment, and an enzyme treatment, or any combination thereof.

In an embodiment, the method may further include filtering the raw water before the freezing and thawing.

In an embodiment, a filter used in the filtering may have a pore size of less than 25 μm.

In an embodiment, the freezing and thawing of the raw water may be repeatedly performed until the number of microplastic particles in the supernatant of the raw water becomes 1×10¹⁰ pieces/mL or less.

In an embodiment, the freezing of the raw water may be performed at a temperature of 0° C. or lower, and the thawing of the raw water may be performed at room temperature.

In an embodiment, the recovering of microplastic aggregates may be performed using a capture filter with a pore size of 10 to 50 μm.

In an embodiment, the analyzing of the recovered microplastic aggregates may be performed by one of Fourier transform IR spectroscopy (FTIR), Image-FTIR, Raman spectroscopy, and pyrolysis gas chromatography/mass spectrometry, or any combination thereof.

In an embodiment, the analyzing of the recovered microplastic aggregates may be performed by image-FTIR.

According to the embodiments of the present disclosure, an improved microplastic analysis method is provided for measuring the content of microplastics in a water system. The method is capable of accurate measurement of nano-sized microplastic particles in water. The method can be performed with high accuracy in a water system while using simplified equipment and process configuration.

### BRIEF DESCRIPTION OF THE DRAWINGS

FIG. 1 shows a process flow diagram of a method of analyzing microplastic particles in a water system, according to an embodiment of the present disclosure;

FIG. 2 shows an image of the turbidity degree of raw water visible to the naked eye depending on the cycle of freezing and thawing, according to another embodiment of the present disclosure;

FIG. 3 shows a graph illustrating change in the number of microplastic particles per unit volume of supernatant of raw water depending on the cycle of freezing and thawing, according to an embodiment of the present disclosure;

FIGS. 4, 5A to 7A, and 5B to 7B show scanning electron microscope (SEM) images of microplastic aggregates depending on the cycle of freezing and thawing, according to an embodiment of the present disclosure; and

FIGS. 8, 9A to 11A, and 9B to 11B show FTIR images of microplastic aggregates in the raw water depending on the cycle of freezing and thawing, according to an embodiment of the present disclosure.

### DETAILED DESCRIPTION

The purposes, advantages, and features of the present disclosure will become more apparent from the following detailed description and embodiments taken in conjunction with the accompanying drawings, but the present disclosure is not necessarily limited thereto. Additionally, in describing the embodiments of the present disclosure, when it is determined that a detailed description of related known techniques may unnecessarily obscure the present disclosure, the detailed description of the known techniques will be omitted.

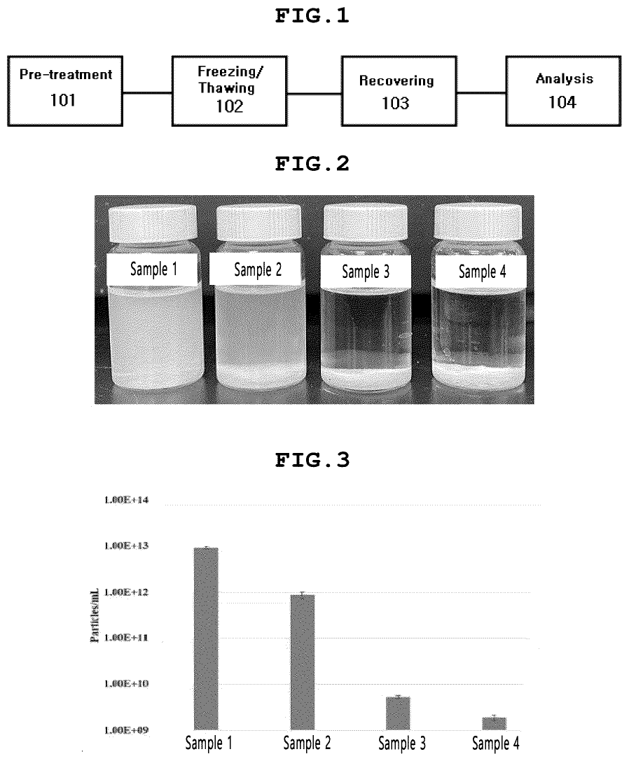

According to an embodiment of the present disclosure, a method of analyzing microplastic particles in a water system includes providing raw water containing microplastic particles; freezing and thawing the raw water at least once to generate microplastic aggregates in the raw water; recovering the microplastic aggregates; and analyzing the recovered microplastic aggregates. The method may be performed in the same order as the flow chart shown in FIG. 1. The raw water which is subjected to the microplastic particles analysis may be untreated water. The raw water which is subjected to the microplastic particles analysis may be partially treated water.

The method includes providing raw water containing microplastic particles. In the present disclosure, “microplastic” refers to a plastic particle in raw water to be introduced into the embodiments of the present disclosure. There is no limit to the type and size of the microplastic particles in the raw water as described in the operations of the embodiments of the present disclosure. For example, the microplastic particles may include not only an amorphous plastic used in disposable packs but also a crystalline plastic such as those used in beverage bottles. The microplastic particles may have a particle size in a range of a few nanometers (nm) to a few centimeters (cm).

In an embodiment, the method may further include removing an impurity on the surface of the microplastic particles in the raw water before the freezing and thawing operations. The operation before the freezing and thawing operations may be referred as pre-treatment 101. In addition to the microplastic particles that are the subject of analysis in the embodiments, the raw water may contain an impurity such as beverages remaining when plastic was used as a beverage bottle. The impurity may stick to the surface of the microplastic particles and act as noise in the subsequent analysis results for microplastic particles. Accordingly, it is preferable to remove the impurity. More specifically, in an embodiment, the method further includes removing an impurity from the surface of the microplastic particles in the raw water before the freezing and thawing. By doing so, the method enables the recovery of pure microplastic particles after removing the impurity from the surface thereof. Using more pure or pure microplastic particles as the subject of the analysis allows obtaining more accurate analysis results.

In an embodiment, the removal of the impurity from the surface of the microplastic particles may be performed by one of a strong acid or strong alkali treatment, an ultrasonic treatment, a plasma treatment, an electrochemical oxidation treatment, and an enzyme treatment, or any combination thereof. The strong acid or strong alkali treatment involves adding a strong acid or strong alkali to the raw water to remove an impurity which is soluble in acid or alkali and is adhering to the surface of the microplastic particles. The ultrasonic treatment and plasma treatment involve separating an impurity from the surface of the microplastic particles through physical methods. The electrochemical oxidation treatment involves immersing an electrode in raw water and applying power to generate free radicals when the impurity on the surface of the microplastic particles is an organic substance, and then oxidizing and removing the impurity through the oxidation reaction of the organic substance by free radicals. The enzyme treatment involves removing the impurity on the surface of the microplastic particles by adding an enzyme to the raw water, the enzyme having substrate specificity for the impurity on the surface of the microplastic particles. Two or more methods for removing the impurity on the surface of the microplastic particles as described above may be used in combination. The methods may selectively remove the impurity without damaging microplastic particles.

In an embodiment, the method may further include filtering the raw water before the freezing and thawing. The analysis target of the embodiments of the present disclosure is microplastic particles in the water. Herein, the microplastic particles may be a plastic with a size of less than 25 micrometers based on the largest dimension. The method may include filtering the raw water using a filter with a predetermined pore size to separate larger plastic particles from the to-be-analyzed microplastic particles.

In an embodiment, the filter used in the filtering may contain pores of less than 25 μm. The method of the present disclosure may include removing microplastic particles with a size of 25 μm or more based on the largest dimension from raw water through filtering using the filter having a pore with a size of less than 25 μm and analyzing microplastic particles only with a size of less than 25 μm. Specifically, the filter used in the filtering may have a pore with a size of less than 10 μm, more specifically less than 1 μm. This allows the particle size of the to-be-analyzed microplastic particles to be set to a narrower range.

The method includes freezing and thawing 102 the raw water at least once to generate microplastic aggregates in the raw water. In the freezing and thawing, the microplastic aggregates are generated in the raw water in a thawed state after the raw water has been subjected to one or more freezing and thawing cycles. Regarding the generated microplastic aggregates, as the cycle of the freezing and thawing increases, the absolute number of microplastic particles in the raw water that form the microplastic aggregates increases. As the number of microplastic particles forming the microplastic aggregates increases, the size of the aggregates also increases. However, the degree of increase in aggregate size may not be significant. Referring to FIG. 2, it is visually confirmed that as the cycle of the freezing and thawing increases, the turbidity of the supernatant separated from the aggregates by precipitation decreases. From this, it is inferred that as the cycle increases, the absolute number of microplastic particles in raw water that form the microplastic aggregates increases. In an embodiment, the freezing and thawing of the raw water may be specifically performed at least once, or 1 to 20 times, 1 to 15 times, and more specifically 5 to 10 times.

In an embodiment, the microplastic aggregates may have a size greater than 25 μm and 10,000 μm or less based on the largest dimension. The microplastic aggregates generated during the freezing and thawing have the same size as above. The microplastic aggregates may be introduced into the analysis without any additional processing or screening. Specifically, the microplastic aggregates may have a size of 100 μm or more and 5,000 μm or less, more specifically 500 μm or more and 3,000 μm or less, based on the largest dimension.

In an embodiment, the freezing and thawing of the raw water may be repeatedly performed until the number of microplastic particles in the supernatant of the raw water becomes 1×10¹⁰ pieces/mL or less. As mentioned above, as the cycle of the freezing and thawing increases, the absolute number of microplastic particles in the raw water that form the microplastic aggregates increases. This means that the number of microplastic particles in the supernatant of the raw water is decreased. By repeating the freezing and thawing as described above until the number of microplastic particles in the supernatant becomes less than 1×10¹⁰/mL, the number of microplastic particles involved in the aggregation operation increases. This may increase the analysis accuracy for the microplastic aggregates. In addition, through the repetition of the freezing and thawing, a supernatant with a greatly reduced concentration of the microplastic particles may be obtained. Therefore, the method is also further applicable to the environmentally friendly water treatment field. Specifically, the freezing and thawing of the raw water may be repeatedly performed until the number of microplastic particles in the supernatant of the raw water becomes 5×10⁹ pieces/mL or less, more specifically, 1×10⁹ pieces/mL or less.

In an embodiment, the freezing of the raw water may be performed at a temperature of 0° C. or lower, and the thawing of the raw water may be performed at room temperature. Specifically, the freezing of the raw water may be performed at a temperature of −20° C. or more and 0° C. or less, or more specifically, −10° C. or more and −5° C. or less. When the freezing of the raw water is performed at a temperature above 0° C., the temperature may exceed the freezing point of the raw water, thereby a freezing reaction may not be carried out smoothly. When the freezing of the raw water is performed at a temperature below −20° C., aggregation may not be effective compared to the energy consumed to lower a temperature, thereby it may be energy inefficient. The thawing of the raw water may be performed at a temperature of 20° C. or more and 40° C. or less, or at 25° C. or more and 35° C. or less. When the thawing of the raw water is performed at a temperature below 20° C., it may take substantial time to thaw the raw water, thereby the time required for the entire process may increase while repeating the freezing and thawing. This may lead to an increase in the overall process cost. When the thawing of the raw water is performed at a temperature exceeding 40° C., the high-temperature effect on the aggregates may not be significant compared to the energy consumed to raise a temperature, thereby it may be energy inefficient.

The method includes recovering the microplastic aggregates 103. The microplastic aggregates generated in the freezing and thawing are precipitated at the bottom of the raw water. In the recovering of the microplastic aggregates, any suitable method may be used provided the precipitated microplastic aggregates are separated and recovered from the supernatant of the raw water.

In an embodiment, the recovering of the microplastic aggregates may be performed using a capture filter with a pore size of 10 to 50 μm. As described above, the microplastic aggregates in an embodiment have a size greater than 25 μm based on the largest dimension. When the capture filter is used, the supernatant of the raw water and microplastic particles may be separated simply by passing the microplastic aggregates through the capture filter. Afterward, the microplastic aggregates may be analyzed without any additional processing. The recovering of the microplastic aggregates may be performed using a capture filter with a pore size of 15 to 40 μm, or a pore size of 20 to 30 μm, or a pore size of 10 to 25 μm.

The method includes analyzing 104 the recovered microplastic aggregates. The analyzing of the microplastic aggregates may include qualitative as well as quantitative analysis. The quantitative analysis means the identification of the size and number of microplastic particles contained in the recovered microplastic aggregates. The qualitative analysis refers to the identification of the types of plastic contained in the recovered microplastic aggregates. In the analysis of the microplastic aggregates, the quantitative analysis and qualitative analysis of the microplastic aggregates may be performed simultaneously.

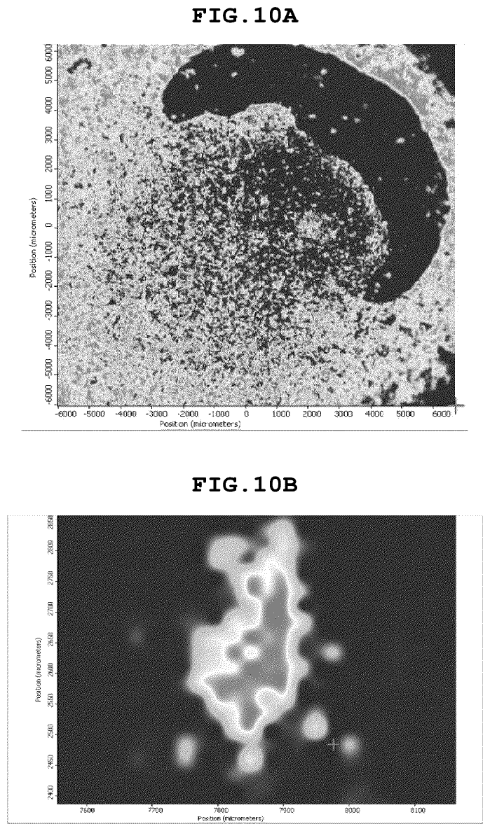

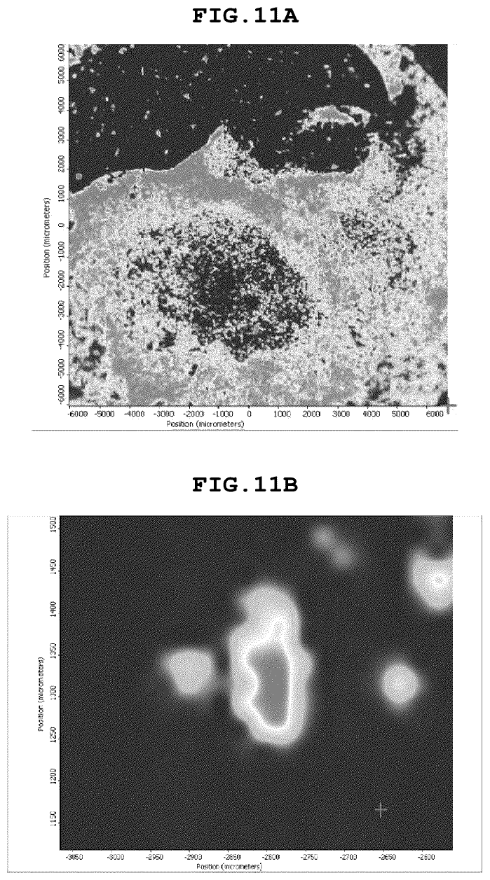

In an embodiment, the analyzing of the recovered microplastic aggregates may be performed by one of Fourier transform IR spectroscopy (FTIR), Image-FTIR, Raman spectroscopy, and pyrolysis gas chromatography/mass spectrometry, or any combination thereof. The Fourier transform IR spectroscopy (FTIR) involves irradiating the microplastic aggregates with white light in the Fourier transform IR region, causing absorption depending on each material, and obtaining a unique spectrum for each material accordingly and then a spectrum for each component of the material. By this method, the type and number of microplastic particles corresponding to the peaks of each spectrum are identified. The Image-FTIR is an analyzing method that identifies the type and number of microplastic particles by imaging the FTIR spectrum as shown in FIGS. 8 to 11B. The Raman spectroscopy is an analyzing method that classifies the types of microplastic particles contained in the microplastic aggregates depending on the scattered degree of incident light by irradiating a laser to the microplastic aggregates. The pyrolysis gas chromatography/mass spectrometry involves breaking down the microplastic aggregates through thermal decomposition and detecting peaks originating from these microplastic particles. By this method, the type of microplastic particles is identified and then information about the mass of each microplastic particle based on the sum of the areas of the peaks is obtained. Through the analyzing methods, it is possible to simultaneously analyze the qualitative and quantitative information of the microplastic particles in the water system. Considering the analysis results, appropriate microplastic removal methods may be introduced for the raw water.

In an embodiment, the analyzing of the recovered microplastic aggregates may be performed by image-FTIR. The image-FTIR is beneficial because analysis results are obtained even with a small amount of sample compared to the other analyzing methods mentioned above. The method is also beneficial because a more detailed analysis is provided of the aggregates, such as microplastic particle size, number, and type of plastic that makes up the microplastic aggregates.

Hereinafter, embodiments of the present disclosure will be further described with reference to specific experimental examples. The examples and comparative examples included in the experimental examples only illustrate embodiments of the present disclosure and do not limit the scope of the appended patent claims. It will be apparent to those skilled in the art that various changes and modifications to the embodiments are possible within the scope and spirit of the present disclosure. Naturally, such changes and modifications fall within the scope of the attached claims.

### Experimental Example 1. Measurement of Change in Extent of Microplastic Aggregation and Number of Microplastic Particles in Supernatant Depending on the Number of Cycles of Freezing and Thawing

A volume of 2 mL of a standard product containing dispersed polystyrene nanoparticles with a size of 100 to 400 nm based on the largest dimension was added to 13 mL of distilled water. The standard was then stirred at a rotation speed of 300 rpm for 5 minutes to obtain a sample of raw water containing microplastic particles. The same process was repeated to prepare three additional samples of the same raw water. As a result, a total of four raw water samples were obtained (samples 1 to 4). Thereafter, freezing the raw water samples at −20° C. for 2 hours and thawing at 25° C. for 2 hours was counted as 1 cycle, Then the cycle was repeated 0, 1, 5, and 10 times for samples 1 to 4, respectively. After completing the respective cycles for the samples, the precipitation of microplastic aggregates in each sample and supernatant turbidity were visually checked. The results are shown in FIG. 2.

In addition, after collecting the supernatant of each sample, nanoparticle tracking analysis (NTA) was performed by using a microscope to measure the number of microplastic particles per unit volume of the supernatant of each sample. The results are shown in FIG. 3.

Referring to FIG. 2, in the sample that did not undergo the freezing and thawing (0 times of cycle), it was confirmed that no precipitation of microplastic aggregates was observed, and the raw water sample itself was very turbid. The sample subjected to one freezing and thawing cycle showed some precipitation, but the supernatant was still observed to be turbid. The samples subjected to 5 and 10 freezing and thawing cycles, respectively, were observed to have their supernatants clear. However, more precipitation was observed in the sample introduced in 10 cycles than in the sample introduced in 5 cycles.

Referring to FIG. 3, it was confirmed that as the cycle of the freezing and thawing increased, the number of microplastic particles contained in the supernatant of the unit volume of the raw water decreased. From this, it was found that as the cycle of the freezing and thawing increased, the number of aggregated microplastic particles increased, and the water quality of the supernatant improved accordingly.

### Experimental Example 2. Comparison of SEM Images and Image-FTIR Analysis Results of Microplastic Aggregates Depending on the Number of Cycles of Freezing and Thawing

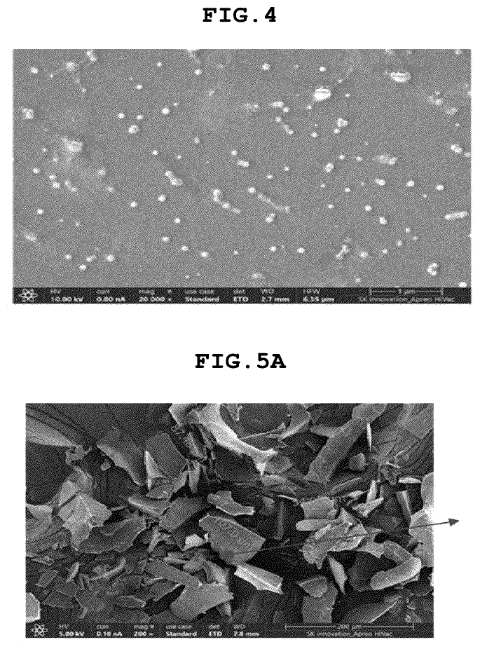

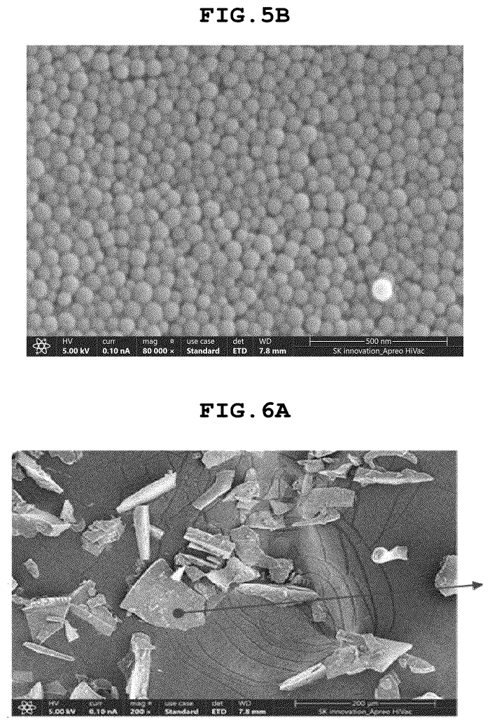

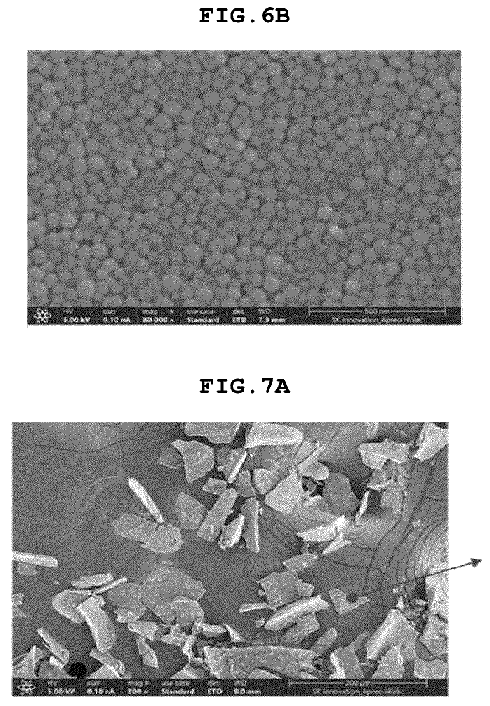

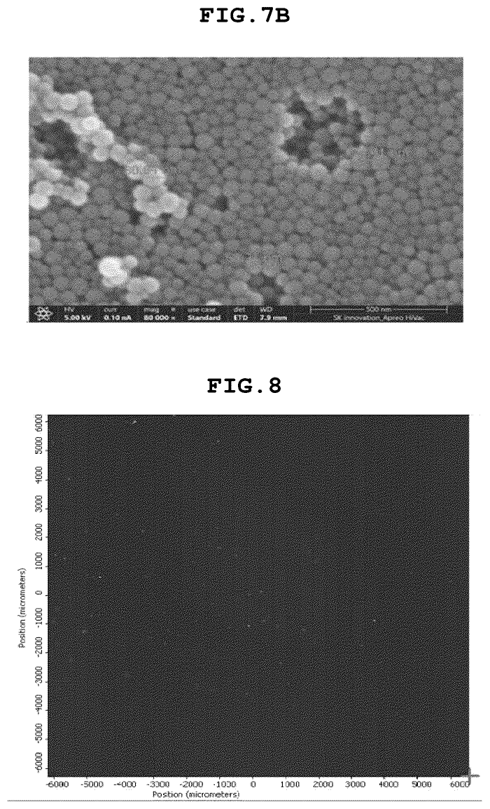

To recover microplastic aggregates by separating the supernatant of raw water, Samples 1 to 4 of Experimental Example 1 were put into a capture filter with a pore size of 25 μm. For Sample 1, no precipitation was performed, and no microplastic aggregates were recovered. For Samples 2 to 4, aggregates were recovered. The raw water of Sample 1 and the recovered microplastic aggregates of Samples 2 to 4 were observed using a scanning electron microscope (SEM). The results were the same as FIGS. 4, 5A, 6A, and 7A, respectively. In addition, in the SEM images of Samples 2 to 4 of FIGS. 5A, 6A, and 7A, when the area indicated by the arrow was observed with the SEM at higher magnification, the results were the same as FIGS. 5B, 6B, and 7B, respectively.

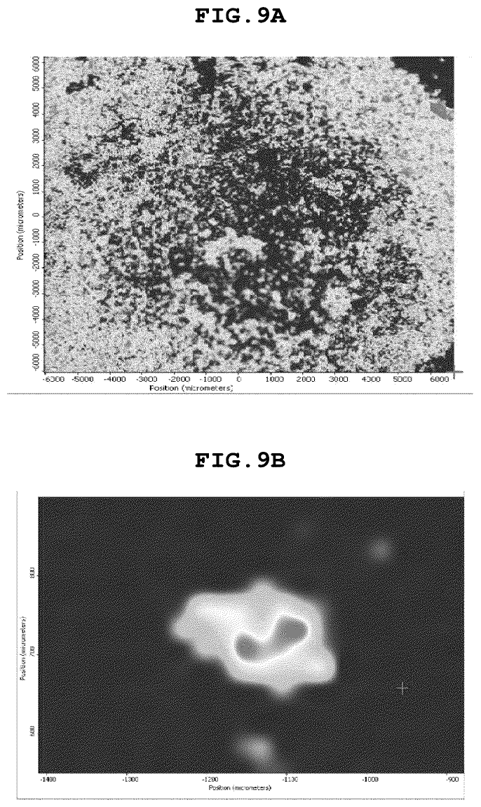

FTIR spectra were obtained for the raw water of Sample 1 and the microplastic aggregates recovered from Samples 2 to 4. Afterward, the FTIR spectra were imaged to obtain FTIR images. FIG. 8 is an FTIR image of the raw water of Sample 1. FIGS. 9A, 10A, and 11A are FTIR images of the recovered microplastic aggregates of Samples 2 to 4. FIGS. 9B, 10B, and 11B are magnified images showing specific portions of the FTIR images of FIGS. 9A, 10A, and 11A. The results of analyzing the size of microplastic aggregates recovered from each of Samples 1 to 4 by using SEM and FTIR images are shown in Table 1.

TABLE 1
Microplastic aggregate size analysis results for Samples 1 to 4.

| | Microplastic aggregate size | |
|---|---|---|
| | (If no aggregates are formed, the | i-FTIR |
| | size of the microplastic particles) | analysis |
| Sample | (nm) | result |
| Sample 1 | 148-367 | Not analyzed |
| Sample 2 | 54,000-107,000 | PS detected |
| Sample 3 | 60,000-119,000 | PS detected |
| Sample 4 | 55,000-155,000 | PS detected |

From Table 1, it was confirmed that as the cycle of the freezing and thawing increased, the size of microplastic aggregates tended to increase.

Additionally, in Sample 1, which did not undergo a cycle of freezing and thawing, the i-FTIR spectrum corresponding to polystyrene (PS) was not detected. In Samples 2 to 4, which were subjected to 1, 5, and 10 cycles of the freezing and thawing, respectively, i-FTIR spectra corresponding to polystyrene (PS) were detected.

The present disclosure has been described in detail above through specific examples. The examples are for specifically describing the embodiments of the present disclosure, however the embodiments of the present disclosure are not limited to these examples only. It should be understood that modifications and improvements may be made by those skilled in the art within the technical concepts and scope of the present disclosure.

All simple modifications or changes to the embodiments of the present disclosure fall within the scope of the present disclosure, and the specific scope of protection of the present disclosure will be made clear by the appended claims. Furthermore, the embodiments may be combined to form additional embodiments.

What is claimed is:

1. A method of analyzing microplastic particles in a water system, the method comprising:
providing raw water containing microplastic particles;
freezing and thawing the raw water at least once to generate microplastic aggregates in the raw water;
recovering the microplastic aggregates; and
analyzing the recovered microplastic aggregates.

2. The method of claim 1, further comprising removing an impurity from the surface of the microplastic particles in the raw water before the freezing and thawing.

3. The method of claim 2, wherein the removal of the impurity from the surface of the microplastic particles is performed by one of a strong acid or strong alkali treatment, an ultrasonic treatment, a plasma treatment, an electrochemical oxidation treatment, and an enzyme treatment, or any combination thereof.

4. The method of claim 1, further comprising filtering the raw water before the freezing and thawing.

5. The method of claim 4, wherein a filter used in the filtering has a pore size of less than 25 μm.

6. The method of claim 1, wherein the freezing and thawing of the raw water is repeatedly performed until the number of microplastic particles in the supernatant of the raw water becomes 1×10¹⁰ pieces/mL or less.

7. The method of claim 1, wherein the freezing of the raw water is performed at a temperature of 0° C. or lower, and the thawing of the raw water is performed at room temperature.

8. The method of claim 1, wherein the recovering of the microplastic aggregates is performed using a capture filter with a pore size of 10 to 50 μm.

9. The method of claim 1, wherein the analyzing of the recovered microplastic aggregates is performed by one of Fourier transform IR spectroscopy (FTIR), Image-FTIR, Raman spectroscopy, and pyrolysis gas chromatography/mass spectrometry, or any combination thereof.

10. The method of claim 9, wherein the analyzing of the recovered microplastic aggregates is performed by image-FTIR.

11. A method of analyzing microplastic particles in raw water comprising water, microplastic particles, and at least one impurity adhering on the surface of the microplastic particles, the method comprising:
removing an impurity from the surface of the microplastic particles in the raw water;
then subjecting the raw water to a cycle of freezing and thawing to generate microplastic aggregates in the raw water;
recovering the microplastic aggregates; and
analyzing the recovered microplastic aggregates.

12. The method of claim 11, wherein the recovering the microplastic aggregates includes microplastic aggregates precipitated at the bottom of the raw water and the precipitated microplastic aggregates are separated from a supernatant using a capture filter with a pore size of 10 to 50 μm.
